(19) United States
(12) Patent Application Publication
(10) Pub. No.: US 2003/0017446 A1
(43) Pub. Date: Jan. 23, 2003

(54) Instruments and methods for creating a tissue microarray

(75) Inventors: Stephen V. Chasse, Franklin, MA (US); Sunny Wai Keung Chu, Waltham, MA (US)

Correspondence Address: FOLEY HOAG, LLP, PATENT GROUP, WORLD TRADE CENTER WEST, 155 SEAPORT BLVD, BOSTON, MA, 02110, US

(73) Assignee: Ardais Corporation

(21) Appl. No.: 10/198,593

(22) Filed: Jul. 18, 2002

Related U.S. Application Data
(60) Provisional application No. 60/306,741, filed on Jul. 20, 2001.

(51) Int. Cl. C12Q 1/00; G01N 33/53; C12M 1/34

(52) U.S. Cl. 435/4; 435/7.1; 435/287.2

(57) ABSTRACT

An instrument for generating a tissue microarray includes a coring tool for coring and removing a sample core from the tissue sample contained in the donor block. An image capture device for capturing a histologic image of a fixed section of tissue sample, corresponding to the tissue sample contained in the donor block, from a sample slide is further provided. A processor is coupled to the image capture device and can receive the histologic image of the fixed section of tissue sample from the image capture device. A display is coupled to the processor for displaying the histologic image. A user interface is coupled to the control system to allow a user to select from the displayed histological image a location for coring and removing a sample core.

### CROSS-REFERENCE TO RELATED APPLICATIONS

The present application claims the benefit of U.S. Provisional Application No. 60/306,741 filed Jul. 20, 2001, which is incorporated herein by reference.

### BACKGROUND

Microarrays of tissue specimens permit the parallel processing of biological samples from a plurality of sources. Such tissue microarrays can comprise hundreds of tissue specimens from multiple sources that are fixed at specific locations in an embedding medium, such as, for example, a block of paraffin. While the use of tissue microarrays can dramatically increase the pace of research, construction of a tissue microarray can be a time consuming, labor-intensive task. Conventional instruments for generating tissue arrays typically do not permit high throughput parallel processing of tissue samples to generate an array. Nor do such instruments allow the operator to select specific micro-anatomic or histologic structures from a tissue sample for automatic inclusion in the tissue microarray.

### SUMMARY

Instruments and methods for generating a tissue microarray are disclosed herein that permit the selection of one or more specific micro-anatomic or histologic structures from a donor tissue sample for inclusion in the tissue microarray. In this manner, for example, a researcher can select specific cells of interest from a tissue sample for inclusion in the tissue microarray. A sample slide containing a section of tissue from the tissue sample can be provided as a reference for the researcher. The tissue section on the sample slide can be correlated with a block of donor tissue such that a researcher can select specific structures of interest from the tissue section on the slide and the corresponding structures of the donor tissue can be automatically removed from the block of donor tissue and placed within the tissue microarray by the instrument.

According to one exemplary embodiment disclosed herein, an instrument for generating a tissue microarray may comprise a coring tool for coring and removing a sample core from the tissue sample contained in the donor block. An image capture device for capturing a histologic image of a fixed section of tissue sample, corresponding to the tissue sample contained in the donor block, from a sample slide is further provided. A processor may be coupled to the image capture device and can receive the histologic image of the fixed section of tissue sample from the image capture device. A display may be coupled to the processor for displaying the histologic image. A user interface may be coupled to the control system to allow a user to select from the displayed histological image a location for coring and removing a sample core.

In certain embodiments, the processor may control the motion and operation of the coring tool to remove a sample core from the tissue sample contained in the donor block based on the location selected by the user. The processor may also control the motion and operation of the coring tool to position the sample core in a hole formed in the recipient block.

The instrument may also include an optional holing tool for forming holes in the recipient block.

In accordance with an exemplary embodiment disclosed herein, a method for generating a tissue array may comprise acquiring an image of a fixed section of tissue sample from a sample slide, displaying the acquired image to a user, providing a user interface to a user to allow the user to select a location on the displayed acquired image to core and remove a sample core, and coring and removing a sample core from the donor block based on the location selected by the user. The exemplary method may also include inserting the sample core into a hole in a recipient block.

### BRIEF DESCRIPTION OF THE DRAWINGS

These and other features of the instruments and methods disclosed herein will be more fully understood by reference to the following detailed description in conjunction with the attached drawings in which like reference numerals refer to like elements through the different views. The drawings illustrate principles of the instruments and methods disclosed herein and, although not to scale, show relative dimensions.

FIG. 1 is a perspective view of an instrument for generating a tissue microarray, illustrating the donor block mounted on a three-axis stage and a retractable slide carriage;

FIG. 2 is a perspective view of an alternative embodiment of an instrument for generating a tissue microarray, illustrating the multi-sample pallet of the instrument holding multiple donor blocks and the associated sample slides;

FIG. 3 is a perspective view of an alternative embodiment of an instrument for generating a tissue microarray, illustrating the multi-sample pallet of the instrument holding multiple donor blocks and a processing station for the donor slides corresponding to the donor blocks;

FIG. 4 is a perspective view of a further embodiment of an instrument for generating a tissue microarray, illustrating the donor block and the sample slide mounted on fixed bases;

FIG. 5 is a perspective view of a donor block mounted on the fixed donor block base of the instrument of FIG. 4 and the corresponding sample slide, illustrating fiducial marks provided on the donor block and the sample slide;

FIG. 6 is a flowchart illustrating a method of generating a tissue microarray; and

FIG. 7 is a schematic diagram of the instrument of FIG. 1.

### DETAILED DESCRIPTION OF EXEMPLARY EMBODIMENTS

To provide an overall understanding, certain illustrative embodiments will now be described; however, it will be understood by one of ordinary skill in the art that the instruments and methods described herein can be adapted and modified to provide instruments and methods for other suitable applications and that other additions and modifications can be made without departing from the scope of the systems and methods disclosed herein.

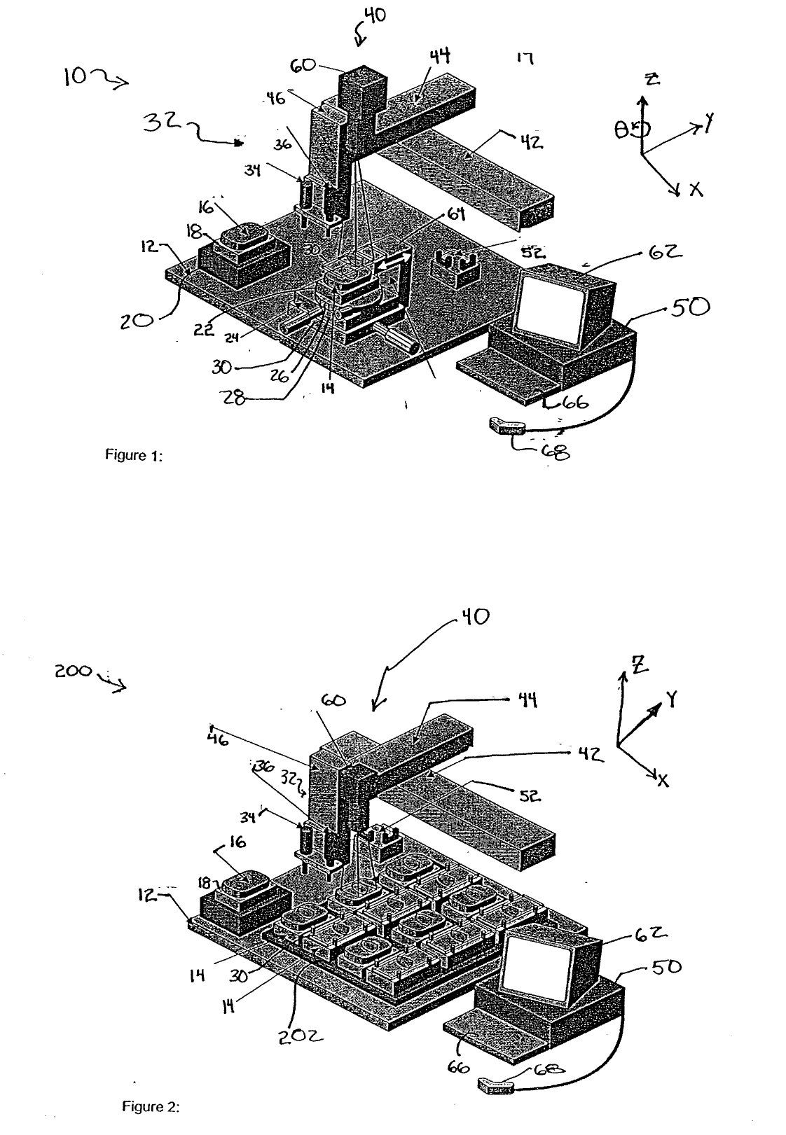

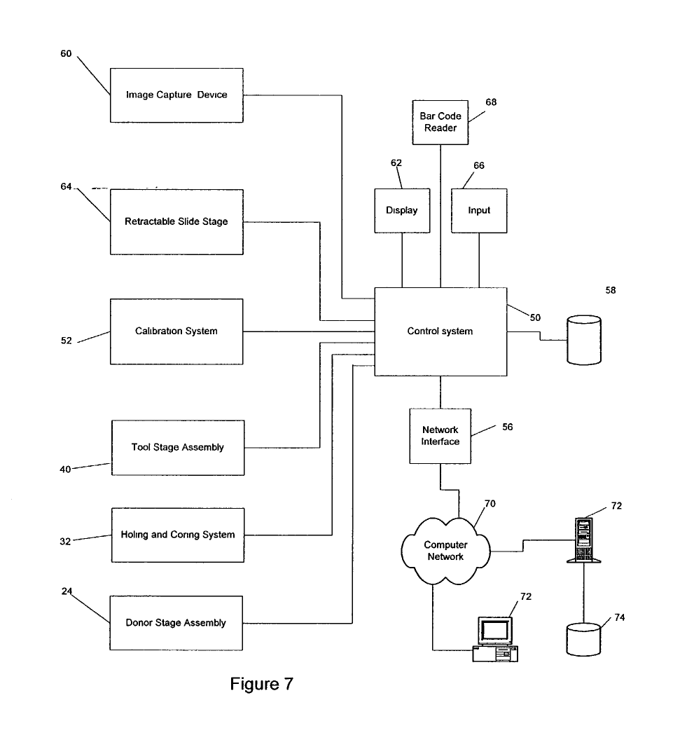

An exemplary embodiment of an instrument 10 for generating a tissue microarray is illustrated in FIGS. 1 and 7. The instrument 10 includes a base 12 for supporting a donor block 14 of sample tissue fixed in an embedding medium such a paraffin or the like. The base 12 additionally supports a recipient block 16, such as, for example, a block of paraffin or other embedding medium. The instrument 10 operates to remove a core of sample tissue from a user-identified location on the donor block 14 and position the core of sample tissue in a user-identified hole formed in the recipient block 16. By repeating this operation, multiple cores of sample tissue can be placed within the recipient block to thereby form an array of sample tissue within the recipient block 16.

The instrument 10 allows an operator to view a histological image of a section of the tissue embedded in the donor block and select the coring locations from the histological image. An alignment process aligns and correlates the orientation of the histological image and the orientation of the tissue sample within the donor block. The instrument 10 automatically removes a core of tissue from the donor block based on the coring location identified by the operator. In this manner, the operator can select micro-anatomical structures of interest from the histological image and the corresponding structures can be cored and removed from the tissue sample and subsequently included in the tissue array.

Continuing to refer to FIGS. 1 and 7, the recipient block 16 is preferably positioned on a support plate 18 or like structure to facilitate transport of the recipient block 16 and minimize handling of the recipient block 16. The recipient block 16 and the support plate 18 may be positioned on a platform 20 provided on base 12. The position and height of the platform are selected to facilitate the holing and coring operations of the instrument as discussed below.

Like the recipient block 16, the donor block 14 is preferably positioned on a support plate 22 or like structure to facilitate transport of the donor block 14 and minimize handling of the donor block 14. Preferably, the tissue sample embedded in the donor block 14 is contrasted against the embedding medium to allow for identification of the boundaries of the tissue sample. Such contrasting can be accomplished by staining the tissue sample or the embedding medium, or by highlighting the boundaries of the tissue sample with ink or other indicia.

The donor block 14 and the support plate 22 may be positioned on an adjustable donor stage assembly 24 that includes a theta (θ) stage 26, a Y-stage 28, and an X-stage 30. The adjustable donor stage assembly 24 allows the donor block 14 to be adjusted in the X, Y, and theta (θ) directions, thus, facilitating the alignment of the tissue sample embedded in the donor block 14 with a reference, such as a section of the tissue sample fixed on a sample slide 30. The stages of the adjustable donor stage assembly 24 may be manually adjustable using micrometer drives or other manually adjustment mechanisms, as illustrated in FIG. 1, or may include automatic, computer controlled servos or other drivers, as illustrated schematically in FIG. 7.

The instrument 10 further includes a holing and coring system 32 having a holing tool 34 for forming holes in the recipient block 16 and a coring tool 36 for coring and removing a core of sample tissue from the donor block 14 and for subsequently inserting the core of tissue into a hole formed in the recipient block 16. The holing tool 34 may be any tool or other mechanism, e.g. a stream of pressurized fluid or energy from a laser or the like, suitable for forming a hole in the embedding medium. The holing tool 34 is preferably a drill that operates to drill or bore a hole within the recipient block 16. The drill can be, for example, a serrated needle, a conventional drill bit, or other structures suitable for forming a hole by rotational contact with the embedding medium. The drill is preferably shaped like a conventional drill bit, i.e., the drill preferably includes spiral or helical grooves extending from the tip of the drill along the shaft of the drill. The spiral grooves provide channels for removal of the embedding medium during the drilling process thereby minimizing damage to the embedding medium surrounding the hole. Alternatively, the holing tool 34 may be a hollow, needle-like punch that operates to punch a hole in the embedding medium, such as the punch described in U.S. Pat. No. 6,103,518, incorporated herein by reference. A suitable ejection mechanism, such as, for example, a stylet or a pneumatic system, for discharging the embedding medium from the lumen of the punch after the hole is formed may also be provided.

The coring tool 36 is preferably a hollow, needle-like punch, as described above, that receives the tissue sample within the lumen of the punch upon insertion into the tissue sample embedded in the donor block 14. This process, referred to herein as “coring,” results in a segment of the tissue sample, referred to herein as the “core,” being held within the lumen of the punch. Upon withdrawal of the coring tool 36 from the tissue sample, the core of tissue sample remains within the lumen of the coring tool, resulting in the removal of the core of tissue sample from the recipient block 16. The coring tool 36 can include an ejection mechanism for discharging the core from the coring tool into, for example, a hole formed in the recipient block 16. The ejection mechanism may be, for example, a reciprocating stylet positioned within the lumen of the coring tool 36. In one preferred embodiment, the ejection mechanism can be a pneumatic system that introduces a pressurized fluid, e.g., air, nitrogen, or water, into the lumen of the coring tool to eject the core of tissue therefrom.

The exemplary instruments described herein and illustrated in FIGS. 1-4 each include a separate holing tool and a separate coring tool. One skilled in the art will appreciate that a combined holing and coring tool may alternatively be utilized. Moreover, additional holing tools and/or coring tools may be provided to increase the throughput of the instrument.

The holing and coring system 32 is preferably mounted on a stage assembly system 40 that allows the holing tool 34 and the coring tool 36 to be moved into a plurality of positions relative to the base 12 of the instrument 10. The stage assembly 40 includes an X-stage 42, a Y-stage 44, and a Z-stage 46 for moving the holing tool 34 and the coring tool 36 along the X-axis, the Y-axis, and the Z-axis, respectively. Each stage of the stage assembly 40 may include a linear driver that is coupled to a computer control system. The computer control system can be, for example, the processor of a personal computer 50 that is programmed with suitable control system software. One skilled in the art will appreciate that other hardware and/or software combinations may be used to implement the control system. The motion of the drivers can be controlled by the computer control system and one or more position sensors, such as, for example, a laser interferometer or the like, coupled to the control system can be used to provide position feedback to the control system. Using an input device 66 associated with the computer 50, the operator can input position commands for execution by the control system. Preferably, the locations of each component positioned on the base 12, e.g., the coring tool 36, the holing tool 34, the recipient block 16, including each hole formed therein, and the donor block 14 are mapped to a coordinate system, referred to herein as the “base coordinate system.” In the exemplary embodiment, the base coordinate system is a Cartesian (X, Y) coordinate system, however, alternative coordinate systems may be employed. The coordinates of each component can be stored in a database 58 coupled to the computer and can be provided to the computer control system to facilitate precise holing and coring operations by the holing and coring system 32. In addition, the computer control system 50 can control the position of the retractable slide stage 64, described below, and, in certain embodiments, the positioning of the donor stage assembly 24.

A calibration system 52 may be provided on the base and coupled to the computer control system 50 to permit calibration of the stage assembly 40. The calibration system 52 is preferably positioned at a known, fixed location on the base 12 and preferably includes one or more sensors, such as, for example magnetic sensors, for accurately ascertaining the X, Y, and Z position of the holing tool 24 and/or the coring tool 36 and transmitting the location information to the computer control system. The calibration system 52 is particularly useful if different holing tools 34 and/or coring tools 36 are used.

An image capture device 60, such as a camera, is mounted to the stage assembly 40 to acquire an image of a fixed section of the tissue sample, corresponding to the tissue sample embedded in the donor block 14, that is mounted to a sample slide 30. The image capture device 60 is coupled to the computer control system 50 and transmits the acquired image to the control system after suitable signal processing, as is known in the art. The image capture device 60 can be, for example, a CCD camera or any other optical sensor or other sensor suitable for acquiring an image and transmitting the image in electronic format to the control system 50. The images acquired by the image capture device 60 may be displayed on a suitable display device 62, such as, for example, a monitor, associated with the computer 50. The image acquired by the image capture device 60 preferably permits microscopic histological examination of the fixed section of tissue sample on the sample slide 30. Moving the image capture device 60 along the Z-axis using the Z-stage 46 can provide course adjustment to the level of magnification of the acquired image. Fine adjustment of the image magnification and the image resolution can be made by adjustment of the image capture device settings, either at the image capture device or through the control system 50.

In the exemplary embodiment illustrated in FIG. 1, the sample slide 32 is mounted on a retractable stage 64 that allows the sample slide 30 to be moved into and out of position above the donor block 14. Preferably, the sample slide 30 is transparent except for the area of the slide 30 containing the tissue section. When the sample slide 30 is positioned over the donor block 14, as shown in FIG. 1, areas of the tissue sample embedded in the donor block 14 may be visible to the image capture device 60 through the sample slide 30 and may be displayed on the display device 62. Using the displayed images as a guide, the orientation of the tissue sample embedded in the donor block 14 can be aligned with the orientation of the tissue section on the sample slide by adjusting the donor block stage assembly 22 until the image of the tissue section overlaps the image of the tissue sample embedded in the donor block 14. This alignment process can facilitate the automatic coring and removal of cores of tissue samples based on the histological examination of the tissue section, as discussed below.

The alignment process can further include mapping a coordinate system, referred to herein as the “image coordinate system” to the histological image of the tissue section. The image coordinate system can be, for example, a Cartesian coordinate system, a polar coordinate system, or other suitable coordinate systems. The image coordinate system is preferably correlated to the base coordinate system, described above, such that each set of coordinates of the image coordinate system corresponds to a set of coordinates of the base coordinate system. In one exemplary embodiment, the image coordinate system can comprise the horizontal and vertical location of each picture element (pixel), or sets of pixels, forming the histological image displayed on the display device 62. Each set of pixel coordinates can be correlated to a set of X, Y coordinates of the base coordinate system. The coordinates of the histological images may be stored in the database 58.

The computer 50 preferably includes one or more input devices 66, such as, for example, a keyboard, a mouse, a trackball or a touch sensitive screen. Using the input device 66, an operator can select a micro-anatomical or histological structure of interest while viewing a microscopic image of the tissue section on the sample slide 30. The image coordinates corresponding to the location of the selected imaged structure can be correlated to the base coordinates of the corresponding structure within the tissue sample embedded in the donor block 14. The stage assembly 40 can then move the coring instrument 36 to the identified set of base coordinates and a core of sample tissue can be removed at the base coordinates. The removed core of sample tissue can subsequently be inserted into a hole formed in the recipient block 16.

The instrument 10 can optionally include a tissue sample tracking system for tracking the tissue sample and the associated sample slide and the recipient block, as well as any data associated therewith. For example, the computer 50 can be provided with a bar code reader 68 for scanning bar. codes associated with the tissue sample and identifying the tissue sample on the donor block, the sample slide, and the recipient block. Data associated with the tissue sample, the sample slide, and the recipient block, referred to herein collectively as the “sample data,” can include, for example, the histological image, the image coordinates of any micro-anatomical structures identified by the operator, the corresponding base (X, Y) coordinates, the hole location within the tissue array, i.e., the recipient block, for each core removed from the tissue sample, and any histological information identified by the operator when viewing the histological image. The sample data may be correlated with the tracking data, e.g., the bar code or other identifier of the tissue sample, the sample slide, and the recipient block, and may be stored in the database 58 coupled to the computer 50.

As best illustrated in FIG. 7, the control system 50 can include a network interface 56 or other suitable communication device to allow the control system to communicate over a computer network 70, such as the Internet, a local area network (LAN), or any other computer network, with other computer systems, computers, or devices capable of accessing the computer network, collectively referred to herein as a “client computer.” In this manner, any data acquired by the instrument can be transmitted over the network 70 to a client computer 72. Thus, for example, the histological image acquired by the image capture device 60 may be transmitted over the network 70 and viewed at a remote location by a client computer 70. Likewise, data, including operating instructions, can be transmitted to the control system 50 over the network 70 from a client computer 72. Thus, coring locations may be selected from a location remote from the instrument 10 and transmitted over the network 70 for execution by the instrument 10.

Moreover, tracking data and sample data stored in the database 58 can be transmitted to a central tissue bank database 74 over the computer network 70. This allows, for example, verification of and validation of the tracking data acquired by the instrument with the bar code reader 68 prior to the holing and coring operation proceeds.

In certain embodiments the recipient block may include preformed holes for receiving sample cores from the donor block. In such embodiments, the holing tool and process of forming holes in the recipient block may be omitted.

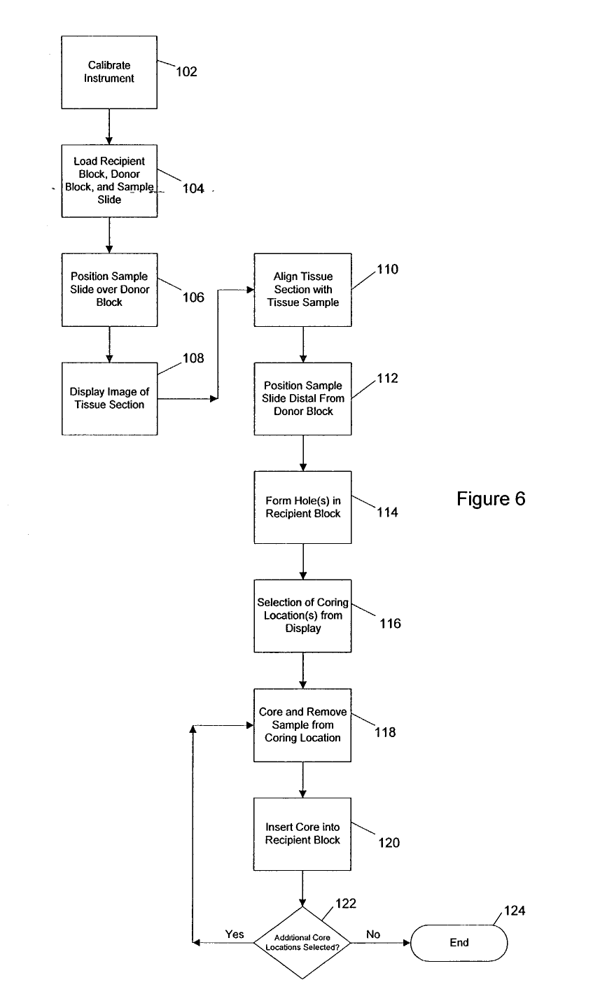

FIG. 6 illustrates an exemplary method for generating a tissue array using the instrument 10 described above. The instrument 10 can optionally be calibrated using the calibration system 52 to accurately ascertain the base coordinates, e.g., the X, Y, and Z position, of the holing tool 24 and/or the coring tool 36, block 102 in FIG. 6. Calibration is optional and may need only be performed when the holing tool 24 and or the coring tool 36 have been changed. The recipient block 16 can be positioned on the stage 20, the donor block 14 can be positioned on the donor stage assembly 24, and the sample slide 30 can be positioned on the retractable slide stage 64, block 104 in FIG. 6. The sample slide 30 can be positioned over the tissue sample within the donor block 14 using the retractable slide stage 64, block 106. The image capture device 60 acquires an image of the tissue section on the sample slide 30, as well as the areas of the donor block 14 visible through the slide 30, block 108 in FIG. 6. The tissue section on the sample slide 30 then can be aligned with the tissue sample on the donor block 14 by adjusting the donor slide stage assembly 24 and using the displayed images as a guide, block 110 in FIG. 6. Upon completion of the alignment process, the sample slide 30 can be moved away from the donor block 14, block 112 in FIG. 6.

One or more holes can be formed in the recipient block 16 using the holing tool 34, block 114 in FIG. 6. The holes in the recipient block 16 may be formed prior to the coring process beginning or alternatively, each hole may be formed separately, prior to the coring and removal of the tissue sample intended for the hole.

The operator can select micro-anatomical structures of the tissue sample for inclusion in the tissue array by viewing the image of the tissue section and using an input device associated with the computer 50 to select structures of interest from the displayed image, block 116 in FIG. 6. In the exemplary method described herein and illustrated in FIG. 6, the operator can select all the coring locations for the tissue sample prior to coring and removal by the coring tool 36.

Continuing to refer to FIG. 6, upon completion of the selection of the coring locations, the holing and coring system 32 can initiate coring and removal of a core of tissue sample at a first one of the selected coring locations, block 116 in FIG. 6. The stage assembly 40 can receive positioning instructions from the computer control system and can position the coring tool 36 at the base coordinates of the first selected coring location. The coring tool 36 can then core and remove a core of tissue from the donor block 14. Once the core is removed, the stage assembly receives further positioning instructions from the computer control system and can position the coring tool 36 at the base coordinates of a hole formed in the recipient block 16. The coring tool 36 can then insert the core of tissue into the hole formed in the recipient block, block 120 in FIG. 6. The control system then queries whether there are additional operator selected coring locations, block 122 in FIG. 6. If there is an additional operator selected coring location, the coring and removal process, block 118 in FIG. 6, and the insertion step, block 120 in FIG. 6, can be repeated for the additional coring location. If not, the process can terminate, block 124 in FIG. 6.

The exemplary method described above in connection with FIG. 6 is provided for illustration and not limitation. One of ordinary skill in the art will appreciate that one or more additional steps may be performed and one or more of the described steps may be omitted without departing from the scope of the present disclosure. Moreover, the order of the steps described above is provided for illustration and not limitation. One of ordinary skill in the art will appreciate that the order of illustrative steps may be varied without departing from the scope of the present disclosure. For example, after each selection or after a set of selections by the operator, block 116 in FIG. 6, and before another selection by the operator, the a core of tissue sample may be cored and removed from the selected location, block 118 in FIG. 6, and inserted into the recipient block 16, block 120 in FIG. 6.

FIG. 2 illustrates an alternative embodiment of an instrument 200 for generating a tissue array. The instrument 200 includes a donor block pallet 202 for supporting a plurality of donor blocks 14 and a plurality of sample slides 30. Preferably, each donor block 14 is positioned adjacent the sample, slide 30 containing the tissue section corresponding to the tissue sample embedded in the donor block 14. The donor block pallet 202 facilitates high throughput generation of tissue arrays by permitting tissue samples from multiple donor blocks 14 to be inserted into the recipient block 14 without necessitating frequent placement of additional donor blocks on the base 12. Although six sets of donor blocks 14 and sample slides 30 are illustrated in FIG. 2, one of ordinary skill in the art will appreciate that any number of donor blocks and sample slides may be provided on the donor block pallet 202 in any configuration.

Alignment of the tissue section on a sample slide 30 with the tissue sample embedded in the corresponding donor block 14 may occur during mounting of the donor blocks and the sample slides to the, donor block pallet 202. During operation, the tissue section on each sample slide can be displayed on display device 62 and the operator can select coring locations for each donor block 14 on, the donor block pallet 202. In accordance with one embodiment, the selection process can be completed prior to the initiation of the tissue coring and removal process. The holing and coring system 32 can then automatically core and remove tissue samples from each donor block 14.

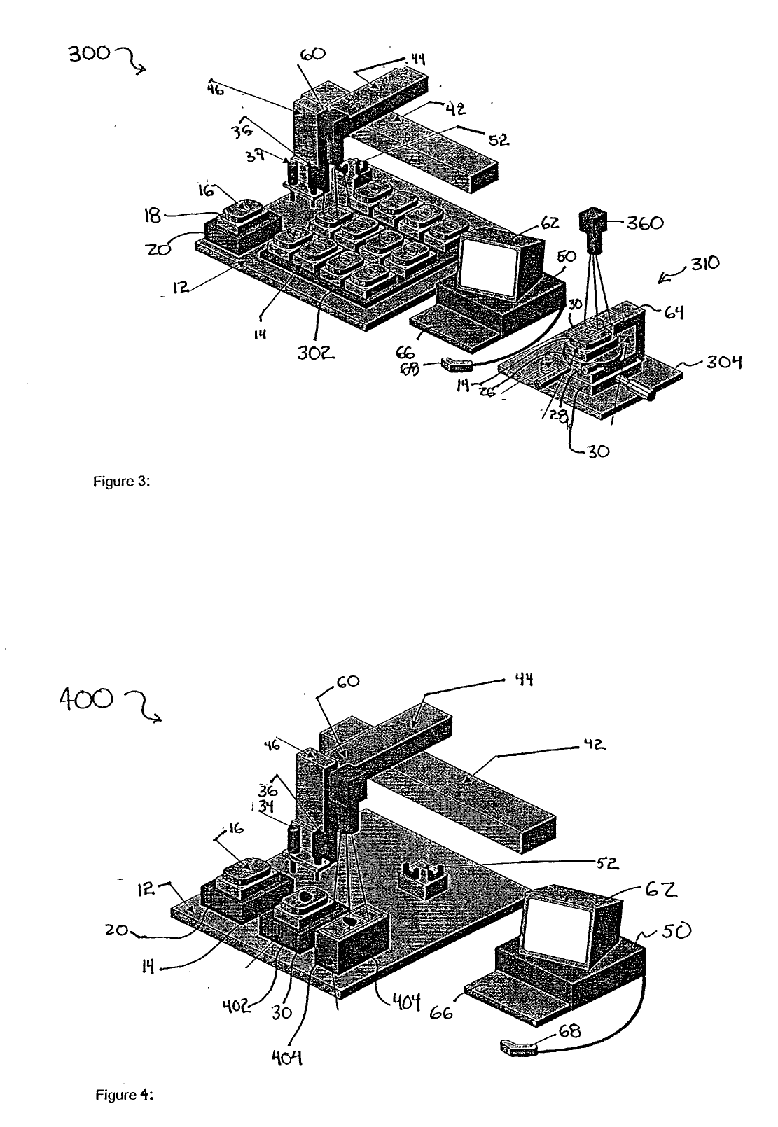

FIG. 3 illustrates a further alternative embodiment of an instrument 300 for generating a tissue array. The instrument 300 includes a donor block pallet 302 for supporting a plurality of donor blocks 14. Like the donor block pallet 202 associated with the instrument 200 described above in connection with FIG. 2, the donor block pallet 302 facilitates high throughput generation of tissue arrays by permitting tissue samples from multiple donor blocks 14 to be inserted into the recipient block 14 without necessitating frequent placement of additional donor blocks on the base 12. Although 12 donor blocks 14 are illustrated in FIG. 3, one of ordinary skill in the art will appreciate that any number of donor blocks may be provided on the donor block pallet 302.

The instrument 300 includes a separate imaging and alignment system 310 for aligning the tissue section on the sample slide 30 with the tissue sample on the donor block 14 and for capturing a histological image of the tissue section. The imaging and alignment system 310 includes a second image capture device 360 for acquiring an image of the tissue section on the sample slide 30.

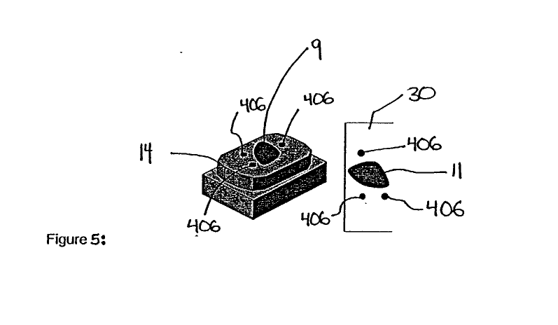

FIGS. 4 and 5 illustrate a further exemplary embodiment of an instrument 400 for generating a tissue array. The instrument 400 includes a fixed donor platform 402 for supporting the donor block 14 and a fixed slide platform 404 for supporting the sample slide 30. The sample slide 30 and the donor block 14 preferably include fiduciary marks 406 that facilitate alignment of the tissue section 11 with the tissue sample 9. During the alignment process, the image of the tissue section 11 and the image of the tissue sample 9 can be aligned by superimposing the fiduciary marks 406 on the slide 30 with the fiduciary marks 406 on the donor block 14. Although illustrated in connection with the embodiment of FIG. 4, one skilled in the art will appreciate that one or more fiduciary marks may be employed with any of the instruments or methods disclosed herein.

One or more fiduciary marks 404 may be added to the donor block 14 prior to taking a section of the donor block 14 to create the a sample slide 30. The material forming the fiduciary marks 404 is preferably selected to have a color in contrast to the color of the donor block 14 and is also preferably selected to adhere to the sample slide 30 during processing. Suitable materials for the fiduciary marks 404 may include, for example, mouse or other animal tissue or silicon. In the case of mouse or other animal tissue, liver tissue is particularly suited because of its generally red color. In the case of silicon, the silicon may be stained to provide the desired contrast.

The process of creating the sample slide 11 from the donor block 30 may cause the tissue section 11 to expand or otherwise change size or shape. The use of fiduciary marks 404 may facilitate alignment of the expanded tissue section 11 with the tissue sample 9 on the donor block 14 by providing reference points for compensating for the expansion of the tissue section 11.

While the instruments and methods disclosed herein have been particularly shown and described with references to exemplary embodiments thereof, it will be understood by those skilled in the art that various changes in form and details may be made therein without departing from the spirit and scope of the disclosure. Those skilled in the art will recognize or be able to ascertain using no more than routine experimentation, many equivalents to the exemplary embodiments described specifically herein. Such equivalents are intended to be encompassed in the scope of the present disclosure.

1. An instrument for generating a tissue array comprising:
a coring tool for coring and removing a sample core from a tissue sample contained in a donor block;
an image capture device for capturing an image of a fixed section of tissue sample from a sample slide, the fixed section of tissue sample corresponding to the tissue sample contained in the donor block;
a processor coupled to the image capture device and receiving the image of the fixed section of tissue sample from the image capture device,
a display coupled to the processor to display the captured image; and
a user interface coupled to the processor to allow a user to select from the displayed captured image a location for coring and removing a sample core.

2. The instrument of claim 1, wherein the processor is coupled to the coring tool, the processor controlling the motion and operation of the coring tool to core and remove a sample core from the tissue sample contained in the donor block based on the location selected by the user.

3. The instrument of claim 2, wherein the processor controls the motion and operation of the coring tool to position the sample core in a hole formed in the recipient block.

4. The instrument of claim 1, further comprising
a holing tool for forming holes in the recipient block.

5. The instrument of claim 4, wherein the holing tool is a drill.

6. The instrument of claim 4, wherein the holing tool is a hollow punch having an ejection mechanism for ejecting material from within the punch.

7. The instrument of claim 1, wherein the coring tool is a hollow punch having an ejection mechanism for the core of tissue sample from within the punch.

8. The instrument of claim 7, wherein the ejection mechanism is a reciprocating stylet positionable within a lumen of the punch.

9. The instrument of claim 7, wherein the ejection mechanism is a pneumatic system for introducing a pressurized fluid into a lumen of the punch.

10. The instrument of claim 1, further comprising
a holing tool for forming holes in the recipient block
a stage assembly for moving at least one of the holing tool and coring tool relative to at least one of the donor block and the recipient block.

11. The instrument of claim 10, wherein the stage assembly comprises
an X-stage, a Y-stage, and a Z-stage.

12. The instrument of claim 10, wherein the stage assembly is coupled to the processor and receives motion control signals from-the processor.

13. The instrument of claim 10, wherein the image capture device is mounted to the stage assembly.

14. The instrument of claim 1, wherein the image capture device is a CCD camera.

15. The instrument of claim 1, further comprising a stage for moving the sample slide into a position above the donor block to facilitate alignment of the sample slide with the donor block.

16. A method for generating a tissue array comprising:
acquiring an image of a fixed section of tissue sample from a sample slide, the fixed section of tissue corresponding to a tissue sample contained in a donor block;
displaying the acquired image to a user;
providing a user interface to a user to allow the user to select a location on the displayed acquired image to core and remove a sample core; and
coring and removing a sample core from the donor block based on the location selected by the user.

17. The method of claim 1, further comprising
inserting the sample core into a hole in a recipient block.

18. The method of claim 1, further comprising
forming a hole in a recipient block for receiving a sample core.

19. The method of claim 1, further comprising
aligning the acquired image of the tissue section with the tissue sample contained in the donor block.

20. A method for generating a tissue array comprising:
acquiring an image of a fixed section of tissue sample from a sample slide, the fixed section of tissue corresponding to a tissue sample contained in a donor block;
aligning the acquired image of the tissue section with the tissue sample contained in the donor block
displaying the acquired image to a user;
providing a user interface to a user to allow the user to select a location on the displayed acquired image to core and remove a sample core;
coring and removing a sample core from the donor block based on the location selected by the user; and
inserting the sample core into a hole in a recipient block.

21. An instrument for generating a tissue array comprising:
a base for supporting a donor block containing a tissue sample and a recipient block;
a holing and coring system for forming holes in the recipient block, coring and removing a sample core from the tissue sample contained in the donor block, and inserting the sample core into a hole formed in the recipient block;
an image capture device for capturing an image of a fixed section of tissue sample from a sample slide, the fixed section of tissue sample corresponding to the tissue sample contained in the donor block;
a computer control system coupled to the image capture device and receiving the image of the fixed section of tissue sample from the image capture device, the computer control system including a display for displaying the captured image; and
a user interface coupled to the control system to allow a user to select from the displayed captured image a location for coring and removing a sample core, the computer control system operating to instruct the holing and coring system to core and remove a sample core from the tissue sample contained in the donor block based on the location selected by the user.
